(19) United States
(12) Patent Application Publication
(10) Pub. No.: US 2024/0110243 A1
(43) Pub. Date: Apr. 4, 2024

(54) Biomarkers Predictive of Atopic Dermatitis that Facilitate Prevention and/or Treatment of the Onset Atopic Dermatitis

(71) Applicants: Johnson & Johnson Consumer Inc., Skillman, NJ (US); University College Cork – National University of Ireland, Cork (IE)

(72) Inventors: Georgios Stamatas, Issy-Les-Moulineaux (FR); Thierry Oddos, Clamart (FR); Takahiro Sato, Raritan, NJ (US); Jonathan Hourihane, Cork (IE); Alan D. Irvine, Dublin 6W (IE); Richard A. Insel, Rochester, NY (US)

(21) Appl. No.: 18/374,074

(22) Filed: Sep. 28, 2023

Related U.S. Application Data
(60) Provisional application No. 63/413,009, filed on Oct. 4, 2022.

(51) Int. Cl. C12Q 1/6883; C12Q 1/6869

(52) U.S. Cl. CPC C12Q 1/6883; C12Q 1/6869; C12Q 2600/106; C12Q 2600/158

(57) ABSTRACT

A method to predict a propensity of an infant to develop atopic dermatitis. The method includes observing the expression of S100A8/A9 on a skin area of an infant; comparing the expression to determined standard, wherein the determined standard is ascertained by measuring a level of the biomarker in a subject or pool of subjects who have demonstrated an absence of atopic dermatitis; and determining the propensity of an infant to develop atopic dermatitis, wherein an increase in the expression as compared to the determined standard indicates propensity of the infant to develop atopic dermatitis.

### CROSS-REFERENCE TO RELATED APPLICATIONS

This application claims the benefit of U.S. Provisional Patent Application No. 63/413,009 filed on Oct. 4, 2022, the disclosure of which is incorporated herein by reference in its entirety.

### FIELD OF THE INVENTION

The invention relates to factors that can be used to predict the propensity of an infant to develop atopic dermatitis. The invention also relates to methods of using the factors to evaluate the potential of a skin treatment regimen, ingredient and/or composition to prevent and/or treat atopic dermatitis in infants.

### BACKGROUND

The skin is a set of cells and macromolecules grouped together in the form of a resistant and flexible tissue which covers the entire body. It is made up of two joined layers, the epidermis and the dermis, and associated subcutaneous tissues.

The main function of the skin is to establish a protective barrier against environmental insults while allowing some exchanges between the internal and external environment. The barrier function is particularly important in limiting epidermal water loss. This function is provided chiefly by the corneal layer (stratum corneum), the uppermost layer of the epidermis, composed of flattened, anucleate cells called corneocytes. The watertightness of this “brick wall” is provided by an intercellular cement composed of specific lipids (cholesterol, cholesterol sulphate, free fatty acids and ceramides). The regenerative capacity of the epidermis is conferred by adult stem cells which allow regular replacement of the differentiated cells eliminated during keratinization. This process is particularly crucial for barrier function maturation and maintenance.

Adaptation to extrauterine life is a process which begins at birth and continues throughout the first year of life. The first months of postnatal life are a period of structural and functional reorganization of the skin allowing physiological adaptation to the extrauterine environment. For example, the immaturity of newborn skin is highlighted by the difference in the structure and molecular composition of the stratum corneum compared with that of adults. These are incomplete and thus continue to develop for at least the first 12 months after. In addition, the results of clinical studies have suggested that infant skin presents a certain immaturity in its ability to capture water and regulate related mechanisms. Moreover, these studies have shown that the epidermal barrier organizes structurally from birth to 2 years of age and is therefore not completely mature during this period. This helps to explain the fragility of infants' and young children's skin and its susceptibility to chemical, physical and microbial attacks.

In addition, incomplete skin maturation can have significant clinical consequences. It is therefore important to allow the skin to be constructed and to develop properly and harmoniously, otherwise its functional and structural organization could be compromised. In this respect, it is crucial to preserve the barrier function and the renewal capacity of the epidermis.

Thus, the immaturity of the barrier and of the mechanisms regulating hydration in a baby's skin contributes to make it even more vulnerable to pathological situations such as atopic dermatitis.

Atopic dermatitis is one of the most common chronic diseases in the population. It is characterized by a set of clinical signs, the most important of which are pruritus and eczematous lesions, which may be acute, subacute or chronic. It almost always begins in infants or young children, while the barrier is structurally and functionally organizing itself. Atopic dermatitis usually begins at around three months of age, but sometimes in the first few weeks of life. It progresses in alternating relapse and remission phases. Depending on the child and the severity of the condition, it may last from several months to several years. A substantial percentage will persist into adulthood.

Atopic dermatitis is, first and foremost, a chronic inflammatory dermatological disease combining impairment of the skin barrier and skin inflammation. In a first sensitization phase, the skin barrier defect allows allergens to penetrate through the skin. Allergens that penetrate the upper layers of the epidermis are processed (internalized) by epidermal Langerhans cells and dermal dendritic cells. Langerhans cells are antigen-presenting cells that are able to capture skin antigens, prepare them and present them to T lymphocytes. This presentation along with direct activation of epidermal alarmins such as IL-33, IL-25 and TSLP lead to activation of the Th2 response, which results in the production of inflammatory cytokines such as IL-4, IL-5, and IL-13.

In all periods of activity of the disease, bacterial or viral skin superinfections are the most common complications. The skin of atopic dermatitis patients is highly susceptible to secondary infections, which then tend to become more widespread. For example, the bacterium Staphylococcus aureus is a major cause of skin infections. It commonly colonizes the skin of atopic dermatitis patients, whereas it is only transiently present on healthy skin. The bacterium then secretes virulence factors that further reduce the barrier function, exacerbating the disease and contributing to its chronicity. In addition, S. aureus is usually found in atopic dermatitis patients in the form of homogeneous biofilms, a form resistant to host defenses and treatments.

Various conventional solutions include methods for assessing the effectiveness of a C7 sugar or derivative thereof in the prevention and/or treatment of at least one deficiency of the skin barrier of a subject, methods for evaluating the in vitro efficacy of formulations in preventing the effects of dehydration on children's skin, and methods for evaluating the effects of a product on epidermal lipogenesis that includes applying the product to the surface of a skin equivalent, measuring the variation of a marker of epidermal lipids, then making a comparison with a similar measurement for a control sample. Other methods include in vivo methods for measuring the binding of chemical compounds or mixtures of compounds to skin constituents.

Some traditional examples of treating sensitive skin, irritated skin, reactive skin, atopic skin, pruritus, ichtyosis, acne, xerosis, atopic dermatitis, cutaneous desquamation, skin subjected to actinic radiation, or skin subjected to ultraviolet radiation include administering an effective amount of a composition comprising furan lipids of plant oil and thereby increasing synthesis of skin lipids.

Example methods of identifying a rinse off personal care composition that includes: (a) generating one or more control skin profiles for two or more subjects; (b) contacting at least a portion of skin of the subjects with a rinse-off test composition, rinsing the test composition off the portion of skin, extracting one or more skin samples from each of the subjects, and generating from the extracted samples one or more test profiles for the subjects; (c) comparing the one or more test profiles to the one or more control profiles and identifying the rinse-off test composition as effective for improving the stratum corneum barrier in a human subject who shows (i) a decrease in one or more inflammatory cytokines, (ii) an increase in one or more natural moisturizing factors, (iii) an increase in one or more lipids, and (iv) a decrease in total protein. Similarly, some emollient use has been correlated with an increased richness and a trend toward higher bacterial diversity as compared to no emollient use in infants at risk for developing atopic dermatitis.

In some examples, microbial richness has been shown to be significantly greater with infant wash and lotion than with wash alone. For example, both cleansing alone and cleansing and emollient regimens have been shown to be well tolerated; skin pH remained slightly acidic throughout the study in each regimen; no significant changes for dryness, redness/erythema, rash/irritation, tactile roughness or total score of objective irritation or for overall skin appearance, in either group vs. baseline at any timepoint; an increase in microbial richness seen by 2 and 4 weeks with wash and by 4 weeks with addition of lotion; by 4 weeks use, lotion use increased richness more than wash alone; mild infant wash+lotion routine may best help improve microbial richness, which may contribute to overall skin barrier health by providing the right environment for healthy skin microbes to flourish.

Additional studies have shown that thymus and activation-regulated chemokine (TARC), an immune biomarker in babies, could help predict the onset and severity of pediatric atopic eczema, or atopic dermatitis (AD) in newborns. For example, tape strips used on the back of patients' hands or in between their shoulder blades have been used to analyze skin cells for immune biomarkers, showing that both term and preterm babies with elevated levels of TARC at 2 months were found to be more than twice as likely to develop eczema by the age of 2 years. A positive correlation has been identified between the level of TARC and the severity of the eczema; the prevalence of the disorder was 34.6% among term children and 21.2% among pre-term children. TARC levels were elevated in children with AD onset before 6 months of age and in children with AD onset between 6 and 24 months of age when compared to children who did not develop AD. The investigators reported that interleukin (IL)-8 and IL-18 were also associated with moderate-to-severe eczema.

To date, there is no cure for atopic dermatitis. Treatments are primarily local, the aim of which is to improve symptoms and control disease progression. As noted above, in particular, the daily use of emollients is essential to restore and protect the damaged skin barrier. Many different emollients are available on the market. However, the precise mechanisms by which they exert their beneficial effects are insufficiently understood. There thus remains a need to further understand the mechanisms and to select effective, well-tolerated emollients to not only treat, but hopefully also prevent atopic dermatitis.

### SUMMARY OF THE INVENTION

The invention relates to factors that can be used to predict the propensity of an infant to develop atopic dermatitis. In accordance with the invention, the method comprises:
a) observing expression of S100A8/9 alone or in combination with other biomarkers on the skin surface of an infant;
b) comparing the expression to a determined standard, wherein the determined standard is ascertained by measuring a level of the biomarker in a subject or pool of subjects who have demonstrated an absence of atopic dermatitis; and
c) determining the propensity of the infant to develop atopic dermatitis, wherein an increase in the expression as compared to the determined standard indicates a propensity of the infant to develop atopic dermatitis.

The infant may be from about 1 week of age to about 12 months of age; from about 2 weeks of age to about 6 months of age; from about one month of age to about 3 months of age; from about 8 weeks of age to about 2 months of age; and about 8 weeks of age.

The skin may be selected from face skin; extremity skin and/or body skin. The skin may be from the cheek. The skin may be from the elbow.

The method may also include:
a step of observing mutation status of FLG using genetic material from the infant to determine FLG genotype of the infant, wherein the FLG genotype is selected from FLG wild type genotype and FLG loss-of-function genotype; and
observing expression of S100A8/9 alone or in combination with other biomarkers on the skin surface of the infant if the infant is determined to have the FLG wild type genotype.

Also in accordance with the invention, a method for evaluating the efficacy of a skin treatment regimen, ingredient and/or composition to treat atopic dermatitis on an infant, comprises:
measuring the level of S100A8/9 alone or in combination with another biomarker on an area of skin of an infant prior to application of the skin treatment regimen, ingredient and/or composition;
applying the skin treatment regimen, ingredient and/or composition to the area of skin for a period of time;
measuring the level of S100A8/9 on the skin area after the skin treatment regimen, ingredient and/or composition application on the area of skin;
wherein the skin treatment regimen, ingredient and/or composition is beneficial to the skin if the level of the S100A8/9 is less than or equal to the no treatment control.

Skin swabs may be employed for sample collection. This method provides benefits over, for example, tape stripping, which is invasive and destroys the barrier.

These and any other methods, skin treatment regimens, ingredients and/or compositions will be described in more detail below.

### BRIEF DESCRIPTION OF THE DRAWINGS

FIG. 1 are boxplots showing levels of S100A8/9 measured in FLG wt and FLG LoF infants at 8 weeks of age on the elbow using a skin swab and broken down by development of AD at 6 months, 12 months, or 12 months cumulative.

FIG. 2 are boxplots showing levels of IL-36γ measured in FLG wt and FLG LoF infants at 8 weeks of age on the elbow using a skin swab and broken down by development of AD at 6 months, 12 months, or 12 months cumulative.

FIG. 3 is a boxplot showing levels of S100A8/9 measured in FLG wt and FLG LoF infants at 8 weeks of age on the elbow using a skin swab and broken down by no AD development, non-persistent AD, and persistent AD.

### DETAILED DESCRIPTION

### Definitions

As used herein, the following terms shall have the meaning specified thereafter:

“Biomarker” as used herein refers to any biological molecule (gene, protein, lipid, metabolite) that, singularly or collectively, reflect the current or predict future state of a biological system. Thus, as used herein, various biomarkers are indicators of the quality of skin. The ability to prevent and/or treat skin conditions can also be assessed by measuring one or more biomarkers.

“Consumer” as used herein refers to an individual who purchases and/or uses skin treatment regimens, ingredients and/or compositions in accordance with the disclosure. In some instances, therefore, a consumer may be alternately referred to herein as a “user.”

“Control” as used herein means a region of epithelial tissue which has not been contacted with and/or by a regimen, ingredient and/or composition which has contacted the affected surface.

“Cumulative incidence” is the proportion of a population at risk that develops the outcome of interest over a specified time period.

“Effective amount” as used herein means an amount of a regimen, ingredient and/or composition sufficient to significantly induce a positive skin benefit, including independently or in combination with other benefits disclosed herein. This means that the content and/or concentration of active component in the regimen, ingredient and/or composition is sufficient that when the regimen, ingredient and/or composition is applied with normal frequency and in a normal amount, the regimen, ingredient and/or composition can result in the treatment of one or more undesired skin conditions. For instance, the amount can be an amount sufficient to inhibit or enhance some biochemical function occurring within the skin. This amount of active component may vary depending upon, among other factors, the type of regimen, ingredient and/or composition and the type of skin condition to be addressed.

“Emollient” as used herein refers to chemical agents specially designed to make the external layers of the skin (epidermis) softer and more pliable.

“Epidermis” as used herein refers to the outer layer of skin, and is divided into five strata, which include the: stratum corneum, stratum lucidum, stratum granulosum, stratum spinosum, and stratum basale. The stratum corneum contains many layers of dead, anucleated keratinocytes that are essentially filled with keratin. The outermost layers of the stratum corneum are constantly shed, even in healthy skin. The stratum lucidum contains two to three layers of anucleated cells. The stratum granulosum contains two to four layers of cells, held together by desmosomes, and contain keratohyaline granules. The stratum spinosum contains eight to ten layers of modestly active dividing cells that are also held together by desmosomes. The stratum basale contains a single layer of columnar cells that actively divide by mitosis and provide the cells that are destined to migrate through the upper epidermal layers to the stratum corneum. The predominant cell type of the epidermis is the keratinocyte. These cells are formed in the basal layer and exist through the epidermal strata to the granular layer at which they transform into the cells know as corneocytes or squames that form the stratum corneum. During this transformation process, the nucleus is digested, the cytoplasm disappears, the lipids are released into the intercellular space, keratin intermediate filaments aggregate to form microfibrils, and the cell membrane is replaced by a cell envelope made of cross-linked protein with lipids covalently attached to its surface. Keratins are the major structural proteins of the stratum corneum. Corneocytes regularly slough off (a process known as desquamation) to complete an overall process that takes about a month in healthy human skin. In stratum corneum that is desquamating at its normal rate, corneocytes persist in the stratum corneum for approximately 2 weeks before being shed into the environment.

“Epithelial tissue” as used herein refers to all or any portion of the epithelia, in particular the epidermis, and includes one or more portions of epithelia that may be obtained from a subject by a harvesting technique known in the art, including those described herein. By way of example and without being limiting, epithelial tissue refers to cellular fragments and debris, proteins, isolated cells from the epithelia including harvested and cultured cells.

“Filaggrin” (filament aggregating protein) as used herein refers to a filament-associated protein that binds to keratin fibers in epithelial cells. Filaggrin is essential for the regulation of epidermal homeostasis. Within the stratum corneum, filaggrin monomers can become incorporated into the lipid envelope, which is responsible for the skin barrier function. Alternatively, these proteins can interact with keratin intermediate filaments. Filaggrin undergoes further processing in the upper stratum corneum to release free amino acids that assist in water retention. Loss-of-function mutations in the filaggrin gene are among the most common and profound single-gene defects identified to date in the causation and modification of disease. Filaggrin encodes an important epidermal protein abundantly expressed in the outer layers of the epidermis. The critical role of filaggrin in epidermal function underlies the pathogenic importance of this gene in common dermatologic and allergic diseases. The spectrum of such diseases encompasses monogenic disorders of keratinization through complex abnormalities of epidermal transport of lipids and allergens. FLG loss-of-function mutation carriers have a greatly increased risk of common complex traits, including atopic dermatitis (which affects at least 42% of all mutation carriers), contact allergy, asthma, hay fever, and peanut allergy. These genetic variants also influence the severity of asthma and alopecia areata and susceptibility to herpetic infection.

Human-beta-defensin 1 (hβD1) is an antimicrobial peptide constitutively expressed by epithelial cells at mucosal surfaces and in the epidermis.

“Infant” as used herein refers to a human whose age ranges from birth to approximately twelve months of life.

“Inflammatory cytokine” is a type of signaling molecule that is secreted from immune cells and certain other cell types that promotes inflammation. Inflammatory cytokines are predominantly produced by T helper cells (Ths) and macrophages and involved in the upregulation of inflammatory reactions.

Interleukin-1 family (IL-1 family) is a group of 11 cytokines that plays a central role in the regulation of immune and inflammatory responses to infections or sterile insults.

IL-1RA (IL-1 receptor antagonist) is a natural antagonist of family members of IL-1.

Interleukin-36 gamma (IL-36gamma; IL-36γ) is cytokine in the IL-1 family with pro-inflammatory effects.

Linear regression is a linear approach for modelling the relationship between a scalar response and one or more explanatory variables (also known as dependent and independent variables). The relationships are modeled using linear predictor functions whose unknown model parameters are estimated from the data. Such models are called linear models.

“Metabolite” as used herein refers to the intermediate end product of metabolism. The term metabolite is usually restricted to small molecules. Metabolites have various functions, including fuel, structure, signaling, stimulatory and inhibitory effects on enzymes, catalytic activity of their own (usually as a cofactor to an enzyme), defense, and interactions with other organisms (e.g. pigments, odorants, and pheromones). A primary metabolite is directly involved in normal “growth”, development, and reproduction. A secondary metabolite is not directly involved in those processes, but usually has an important ecological function.

“Package” includes any suitable container for personal care regimens, ingredients and/or compositions.

“Personal care composition” as used herein, refers to compositions intended for topical application to the skin. The compositions used in accordance with the present disclosure include topically applied compositions, including leave-on formulations, and rinse-off formulations in which the product is applied topically to the skin and then is subsequently rinsed within minutes from the skin with water, or otherwise wiped off using a substrate with deposition of a portion of the composition. The personal care composition used in accordance with the present disclosure is typically dispensable from a package. Thus, in some embodiments, the dispensing may be by extruding. In some embodiments the package may be a single chamber package, or a multi chamber package, or a set of discrete packages. The personal care compositions used in accordance with the present disclosure can be in the form of liquid, semi-liquid, cream, lotion or gel intended for topical application to skin.

“P-value” is a measure of the probability that an observed difference could have occurred just by random chance. The lower the p-value, the greater the statistical significance of the observed difference. P-value can be used as an alternative to or in addition to pre-selected confidence levels for hypothesis testing. The standard level of significance used to justify a claim of a statistically significant effect is 0.05. The term statistically significant has become synonymous with p≤0.05.

“Rinse-off” ingredients or compositions by which is meant the ingredient or composition is applied topically to skin and then subsequently and immediately (i.e., within minutes) rinsed away with water, or otherwise wiped off using a substrate or other suitable removal means.

“Skin” is divided into three main structural layers, the outer epidermis, the inner dermis, and the subcutaneous tissue.

“Stratum corneum” as used herein, refers to the outermost layer of the epithelia, or the epidermis, and is the skin structure that provides a chemical and physical barrier between the body of an animal and the environment. The stratum corneum is a densely packed structure comprising an intracellular fibrous matrix that is hydrophilic and able to trap and retain water. The intercellular space is filled with lipids formed and secreted by keratinocytes and which provide a diffusion pathway to channel substances with low solubility in water.

“Subject” as used herein refers to a human for whom a regimen, ingredient and/or composition is tested or on whom a regimen, ingredient and/or composition is used in accordance with the methods described herein.

“Substantially free of” as used herein, unless otherwise specified, means that the personal care regimen, ingredient and/or composition comprises less than about 2%, less than about 1%, less than about 0.5%, or even less than about 0.1% of the stated ingredient. The term “free of”, as used herein, means that the personal care regimen, ingredient and/or composition comprises 0% of the stated ingredient. However, these ingredients may incidentally form as a by-product or a reaction product of the other components of the personal care regimen, ingredient and/or composition.

“Test ingredients and/or compositions” as used herein include and encompass purified or substantially pure ingredients and/or compositions, as well as formulations comprising one or multiple ingredients and/or compositions. Thus, non-limiting examples of test ingredients and/or compositions include water, a pharmaceutical or cosmeceutical, a product, a mixture of compounds or products, and other examples and combinations and dilutions thereof.

“Test surfaces” as used herein means a region of epithelia tissue which has been contacted with and/or by a product, such as a consumer product and/or a test regimen, ingredient and/or composition, whereby the contact of the product and/or the regimen, ingredient and/or composition on the epithelia tissue has resulted in some change, such as but not limited to, physiological, biochemical, visible, and/or tactile changes, in and/or on the epithelia tissue that may be positive or negative. In some examples, positive effects caused by regimen, ingredient and/or composition may include but are not limited to, reduction in one or more of erythema, trans-epidermal water loss (TEWL), discoloration of the skin, rash, dermatitis, inflammation, eczema, dandruff, edema and the like. The location of the affected surface will depend upon the regimen, ingredient and/or composition used or the location of some physiological, biochemical, visible, and/or tactile change in and/or on the epithelia tissue.

“Topical application”, “topically”, and “topical”, as used herein, mean to apply the regimen, ingredient and/or composition used in accordance with the present disclosure onto the surface of the skin.

“Treating” or “treatment” or “treat” as used herein includes regulating and/or immediately improving skin appearance and/or feel.

“Young child/children” as used herein refers to a human/humans whose age ranges from approximately twelve months of life to approximately 3 years, or approximately 5 years, or approximately 7 years of life.

### Examples

The present inventors have identified inflammatory cytokines on the body that can be used to distinguish high-risk infants capable of developing AD within 12 months vs. low risk that will most likely not develop AD.

### STOP AD Trial

STOP AD (Short-term Topical Application for Prevention of Atopic Dermatitis), a randomized, open-label, controlled study designed to investigate the effect of short-term neonatal skin barrier protection using a defined skin treatment regimen and composition on the prevention of atopic dermatitis in high-risk infants, was conducted. (See co-pending U.S. Application Ser. No. 63/327,052; Ni Chaoimh, et al., Parental compliance with an infant moisturization protocol in the first 2 months of life; Lad et al., Can more be done to implement translational weaning advise for new mothers; and Lad et al., Neonatal natural moisturizing factor concentrations in a high-risk cohort with parental history of atopy compared to a reference cohort, each presented at European Academy of Allergy and Clinical Immunology (EAACI) 2020)).

In the study, high-risk infants identified using parental history of atopic disease were randomized to either the defined skin treatment regimen and composition or standard routine care from soon after recruitment in the postnatal ward (approximately 4 day) until eight weeks of age. Both the intervention and standard of care groups were advised to use a standardized bathing routine for the first 2 months.

Skin treatment regimen and composition: twice daily whole-body application of AVEENO® Dermexa Fast & Long-Lasting Balm, which on pack contains the following ingredients: glycerin, aqua, cetearyl alcohol, isocetyl alcohol, dimethicone, cetyl alcohol, Avena sativa kernel flour, Avena sativa kernel extract, Avena sativa kernel oil, caprylic/capric triglyceride, ceramide 3, ethylhexylglycerin, p-anisic acid, sodium cetearyl sulfate, palmitic acid, stearic acid, sodium sulfate, sodium chloride, citric acid, dipotassium phosphate, potassium phosphate, sodium hydroxide, tocopherol, benzyl alcohol, benzoic acid, and potassium sorbate.

Control: Standard routine care, which involved no use of moisturizer for the first two months. Both groups received: AVEENO® Baby Daily Care Gentle Wash, which on pack contains the following ingredients: aqua, glycerin, cocamidopropyl betaine, sodium lauroamphoacetate, coco-glucoside, sodium chloride, hydroxypropyl starch phosphate, Avena sativa kernel flour, Aloe barbadensis leaf juice, Olea europaea leaf extract, Chamomilla recutita extract, Helianthus annuus seed oil, sarcosine, magnesium aspartate, potassium aspartate, polyquaternium-7, polysorbate 20, sodium cocoyl amino acids, acrylates/C10-30 alkyl acrylate crosspolymer, propylene glycol, citric acid, sodium hydroxide, tocopherol, tocopheryl acetate, sodium benzoate, potassium sorbate, sodium sulfite, parfum.

Infants with at least one parent with a positive history of atopic disease (AD, allergic rhinitis or asthma) were eligible for recruitment.

Within approximately 4 days of birth, infants were randomized to either treatment with skin treatment regimen and composition or to standard routine skincare with no moisturizer until 2 months of age. 260 infants participated in the study, including 120 in the intervention group and 140 in the control group.

The study had six visits during the first year of life, within 4 days of birth and at 2, 4 and 8 weeks and 6 and 12 months, involving repeat measurements of weight, trans-epidermal water loss (TEWL) and Raman-derived natural moisturizing factor (NMF) to assess skin barrier function and structure in addition to monitoring of skin health and feeding. A questionnaire on infant health, bathing, feeding and skincare was filled out and skin swabs were taken for microbiome and immune biomarker analysis.

The study's primary outcome was the effect of the intervention on the incidence of atopic dermatitis at 12 months. Secondary outcomes included the effect of the intervention on the incidence of atopic dermatitis at 6 months and the evolution of TEWL and NMF values from 0-12 months.

Skin swabs were taken at baseline and again at 8 weeks and at 12 months. A healthcare worker blind to treatment allocation assessed the presence (yes/no), extent and severity of atopic dermatitis at 6 and 12 months. A DNA sample was taken to test for filaggrin loss-of-function mutations, which are linked to atopic dermatitis risk.

I-SEAL (Insights towards understanding Skin Function in Early Life) involved the collection of skin microbiome and immunity biomarkers within the larger intervention trial STOP AD in order:
to examine the temporal transition of the skin microbiome between birth and 12 months and its influence on the development of atopic dermatitis;
to investigate the impact of the use of the skin regimen and composition in the first two months of life on infant skin microbiota; and
to investigate the dynamics of immunity biomarkers collected from the surface of infant skin in the first 12 months, and to examine associations with atopic dermatitis.

103 participants were diagnosed with atopic dermatitis. The prevalence of atopic dermatitis was 27.3% and 27.9% at 6 and 12 months, respectively, and 17.0% participants met the UK Working Party Diagnostic Criteria, Williams et al., Br J Dermatol. 1994 September; 131(3):406-16. doi: 10.111141365-2133.1994.tb08532.x., at both 6 and 12 months.

### Interim Analysis at Six (6) Months

Of the 262 participants, 260 (120 intervention and 140 control) were included in the interim analysis at 6 months. Two participants, one from the intervention group and one from the control group, were excluded from the analysis due to missing data on atopic dermatitis outcome at 6 months. Baseline characteristics were distributed evenly across study groups. In the total cohort, the point prevalence and cumulative incidence of atopic dermatitis at 6 months was 27.3% and 28.1%, respectively. The cumulative incidence of atopic dermatitis was 18.3% in the intervention group and 36.4% in the control group [relative risk (RR): 0.503, 95% CI: 0.325, 0.779] corresponding to a 50% decreased risk of atopic dermatitis at 6 months in the intervention group.

DNA samples for the analysis of filaggrin gene status were collected using buccal swabs for filaggrin genotyping.

Skin swabs for microbiome and immune biomarker analysis were taken at baseline, at the end of the intervention period (8 weeks) and at 12 months from study participants. Swabs were taken from two skin sites: the cheek and the antecubital fossa (elbow pit). Skin swabs from a subgroup of approximately 30 infants from each study group (control and intervention) were sent to CosmosID, Rockville, MD, USA for analysis at the end of the 12-month assessments.

Swabs taken for microbiome analysis at the end of sample collection at 12 months were analyzed as a single batch for common 16s rRNA gene sequencing.

In addition to the microbiome swabs, samples from the same subgroup identified for microbiome analysis were sent for analysis at the end of sample collection. Samples for the quantification of immunity biomarkers were taken from the skin sites at baseline, 8 weeks and 12 months using specialized swabs soaked in buffer (FibroTx, Tallinn, Estonia). Samples were stored on dry ice until further processing and at −80° C. until shipment to FibroTx for marker analysis using a spot enzyme-linked immunosorbent assay.

The samples were analyzed as follows:
10 cytokines were measured:
   CCL27, CXCL2, hBD1, IL18, IL8, IL1a, IL1RA, IL1b, S100A8/9, IL36g.
Samples were taken from 2 body sites, i.e., elbow and cheek.
There were 2 treatment groups, i.e., A and B. The analysis was done in a blind manner. Groups A and B represent either of intervention or control group.
Samples were taken at 3 time points, i.e., at 0 weeks, 8 weeks and 12 months.
There were 3 AD endpoints, i.e., at 6 months, 12 months and 12 month cumulative incidence.
   43 subjects were in group A and 43 subjects were in group B.
      Group A: 43 (week 0), 43 (week 8), 30 (week 12)
Group B: 43 (week 0), 43 (week 8), 30 (week 12)

Surface cytokine concentrations were measured at 8 weeks of age on the elbow using a skin swab and development of AD was measured at 6 and 12 months (Table 1). Table 1 shows the number of subjects that developed AD at each endpoint.

TABLE 1

| | | | 12 month |
|---|---|---|---|
| | 6 months | 12 months | cumulative |
| Wt | 12 | 22 | 28 |
| LoF | 10 | 11 | 13 |

Table 2 shows biomarker from elbow at week 8 p-values from linear model comparing converters vs. non converters for wild type FLG and loss of function FLG.

TABLE 2

| Elbow Week 8 measurements | | | | | | | |
|---|---|---|---|---|---|---|---|
| wt | | | 12 month | LoF | | | 12 month |
| Biomarker | 6 month | 12 month | cumulative | Biomarker | 6 month | 12 month | cumulative |
| CCL.27 | 1.000 | 1.000 | 1.000 | CCL.27 | 1.000 | 1.000 | 1.000 |
| CXCL.2 | 1.000 | 1.000 | 0.828 | CXCL.2 | 1.000 | 1.000 | 1.000 |
| hBD.1 | 0.082 | 0.993 | 0.862 | hBD.1 | 0.903 | 0.994 | 0.908 |
| IL.18 | 1.000 | 1.000 | 1.000 | IL.18 | 1.000 | 1.000 | 1.000 |
| IL.8 | 1.000 | 1.000 | 1.000 | IL.8 | 1.000 | 1.000 | 1.000 |
| IL.1a | 0.156 | 0.491 | 0.339 | IL.1a | 1.000 | 1.000 | 0.994 |
| IL.1RA | 0.338 | 0.672 | 0.788 | IL.1RA | 1.000 | 1.000 | 1.000 |
| IL.1b | 0.971 | 1.000 | 1.000 | IL.1b | 1.000 | 1.000 | 1.000 |
| S100A8/9 | 0.009 | 0.035 | 0.033 | S100A8/9 | 1.000 | 1.000 | 0.995 |
| IL.36 | 0.984 | 0.018 | 0.038 | IL.36 | 0.881 | 1.000 | 0.978 |

Table 3 shows biomarker from elbow at week 8 p-values from linear model comparing persistent or non-persistent AD converters vs. non converters for wild type FLG and loss of function FLG.

TABLE 3
Elbow Week 8 measurements

| wt | | | LoF | | |
|---|---|---|---|---|---|
| | | non- | | | non- |
| | persistent | persistent | | persistent | persistent |
| Biomarker | vs no AD | vs no AD | Biomarker | vs no AD | vs no AD |
| CCL.27 | 1.000 | 1.000 | CCL.27 | 1.000 | 1.000 |
| CXCL.2 | 1.000 | 0.959 | CXCL.2 | 1.000 | 1.000 |
| hBD.1 | 0.481 | 1.000 | hBD.1 | 0.969 | 1.000 |
| IL.18 | 1.000 | 1.000 | IL.18 | 1.000 | 1.000 |
| IL.8 | 1.000 | 1.000 | IL.8 | 1.000 | 1.000 |
| IL.1a | 0.630 | 0.948 | IL.1a | 1.000 | 1.000 |
| IL.1RA | 0.305 | 1.000 | IL.1RA | 1.000 | 1.000 |
| IL.1b | 1.000 | 0.990 | IL.1b | 1.000 | 1.000 |
| S100A8/9 | p < 0.001 | 0.998 | S100A8/9 | 1.000 | 1.000 |
| IL.36 | 0.332 | 0.403 | IL.36 | 0.997 | 1.000 |

Observations from the above data include:
FLG wt converters have increased cytokine levels at week 8
   S100A8/9 is the most prominent cytokine associated with future development of AD
      Therefore by assessing levels of S100A8/9 patients at higher risk of developing AD can be identified. Such patients can benefit from skin barrier repair treatments. FLG LoF converters have minimal cytokine differences at week 8
   Therefore in this population conversion to AD may be driven by skin barrier function and not skin inflammation.

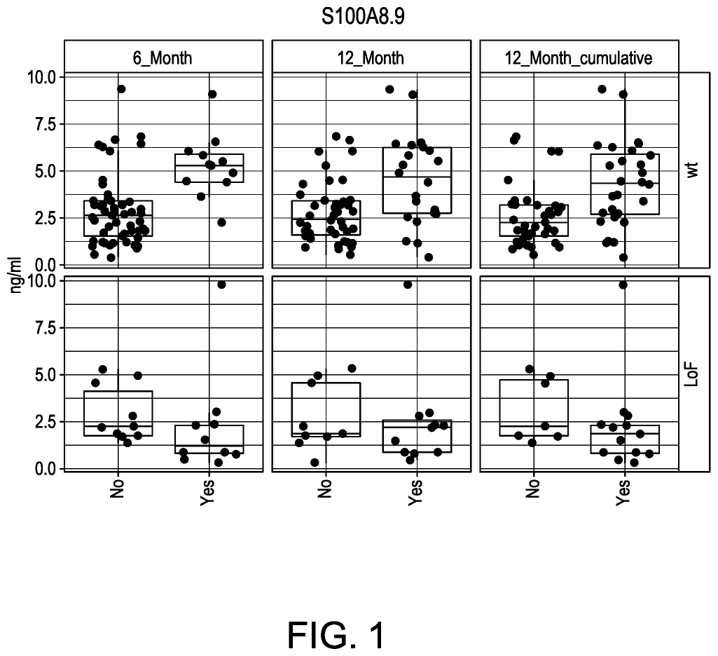

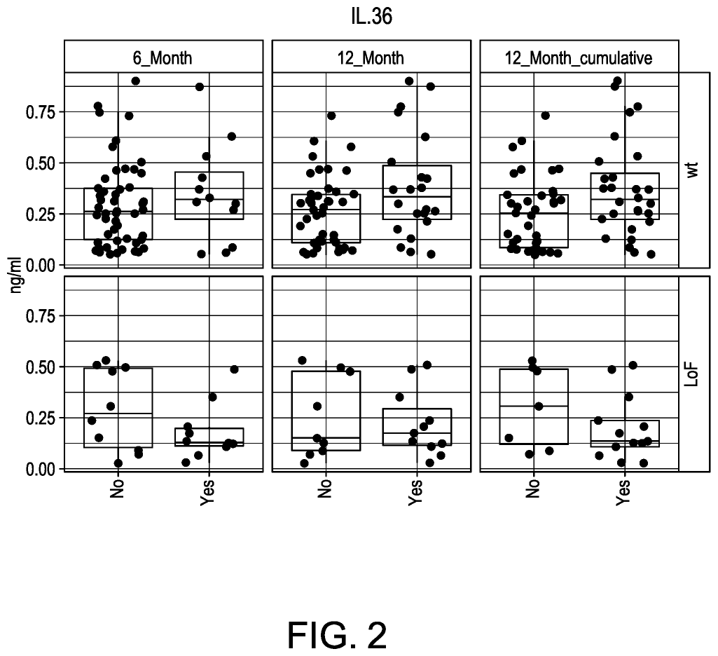

The data were analyzed to test whether cytokine levels could predict development of future AD separately in the FLG wt and FLG LoF groups. In the I-SEAL cohort, there were 65 wt FLG subjects and 20 subjects with FLG LoF mutations. In FLG wt subjects, S100A8/9 was significantly different between converters and non-converters for all 3 endpoints (FIG. 1, Table 2), while IL-36γ was significant for both 12-month endpoints (FIG. 2, Table 2). Other cytokines that had potential signal (p<0.3 at any of the endpoints) in converters include Human β-defensin (hBD)-1 and IL-1a. Subjects with FLG LoF mutations did not have any cytokines that could distinguish between converters and converters (Table 2).

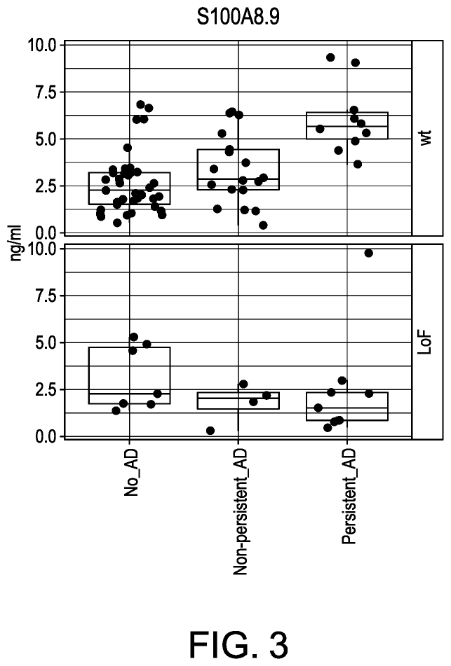

Cytokine levels between non-AD, non-persistent AD (AD at 6 or 12 months only), and persistent AD (AD at both 6 and 12 month end points) was compared. S100A8/9 was significantly different between FLG wt converters and non-converters that developed persistent AD but not in non-persistent AD. (FIG. 3, Table 3). Subjects with FLG LoF mutations did not have any cytokines that could distinguish between converters and non-converters that developed persistent or non-persistent AD (Table 3).

Collectively, these data indicate that cytokine expression in FLG wt subjects measured at 8 weeks of age may predict future onset of AD at 6 and 12 months. In addition, S100A8/A9 measured at 8 weeks of age had a strong prediction of persistent AD compared to non-persistent AD.

It will be understood that, while various aspects of the present disclosure have been illustrated and described by way of example, the invention claimed herein is not limited thereto, but may be otherwise variously embodied according to the scope of the claims presented in this and/or any derivative patent application.

What is claimed is:

1. A method of predicting a propensity of an infant to develop atopic dermatitis, the method comprising:
observing the expression of S100A8/A9 on a skin area of an infant;
comparing the expression to a determined standard, wherein the determined standard is ascertained by measuring a level of the biomarker in a subject or pool of subjects who have demonstrated an absence of atopic dermatitis; and
determining the propensity of an infant to develop atopic dermatitis, wherein an increase in the expression as compared to the determined standard indicates propensity of the infant to develop atopic dermatitis.

2. The method of claim 1, wherein the infant is of an age selected from the group consisting of from about 1 week of age to about 12 months of age; from about 2 weeks of age to about 6 months of age; from about one month of age to about 3 months of age; from about 8 weeks of age to about 2 months of age; and about 8 weeks of age.

3. The method of claim 1, wherein the skin is selected from face skin, extremity skin, and body skin.

4. The method of claim 3, wherein the face skin is from a cheek.

5. The method of claim 3, wherein the extremity skin is from an elbow.

6. The method of claim 1, further comprising:
a step of observing mutation status of FLG using genetic material from the infant to determine FLG genotype of the infant, wherein the FLG genotype is selected from FLG wild type genotype and FLG loss-of-function genotype; and
observing expression of S100A8/9 alone or in combination with other biomarkers on the skin surface of the infant if the infant is determined to have the FLG wild type genotype.

7. A method for evaluating the efficacy of at least one of a skin treatment regimen, ingredient, or composition to treat atopic dermatitis in an infant, comprising:
measuring the level of S100A8/A9 on an area of skin of an infant prior to application of the skin treatment regimen, ingredient and/or composition;
applying the skin treatment regimen, ingredient and/or composition to the area of skin for a period of time; and
measuring the level of S100A8/A9 after the skin treatment regimen, ingredient and/or composition application on the area of skin;
wherein the skin treatment regimen, ingredient and/or composition is beneficial to the skin if the level of the S100A8/A9 is less than or equal to the no treatment control.

8. The method of claim 7, wherein the face skin is from a cheek.

9. The method of claim 7, wherein the extremity skin is from an elbow.

10. The method of claim 7, further comprising:
a step of observing mutation status of FLG using genetic material from the infant to determine FLG genotype of the infant, wherein the FLG genotype is selected from FLG wild type genotype and FLG loss-of-function genotype; and
observing expression of S100A8/9 alone or in combination with other biomarkers on the skin surface of the infant if the infant is determined to have the FLG wild type genotype.
